(19) United States
(12) Patent Application Publication
(10) Pub. No.: US 2022/0008303 A1
(43) Pub. Date: Jan. 13, 2022

(54) OPACIFIER CONCENTRATE AND ITS USE TO MODIFY THE APPEARANCE AND/OR INCREASE OPACITY AND/OR WHITENESS OF AN AQUEOUS COMPOSITION

(71) Applicant: RHODIA OPERATIONS, Aubervilliers (FR)

(72) Inventors: Howard PROKOP, Feasterville, PA (US); Céline ORIZET, Bourg-la-Reine (FR); Ali SAHOUANE, Saint-Maur-des-Fossées (FR); Hocine KABIR, Sérézin du Rhone (FR); Galder CRISTOBAL, SINGAPORE (SG)

(21) Appl. No.: 17/294,677

(22) PCT Filed: Nov. 20, 2019
(86) PCT No.: PCT/EP2019/081939

§ 371 Date: May 17, 2021

Related U.S. Application Data
(60) Provisional application No. 62/769,757, filed on Nov. 20, 2018.
(60) Provisional application No. 62/827,919, filed on Apr. 2, 2019.

(51) Int. Cl. A61K 8/37; A61K 8/42

(52) U.S. Cl. CPC A61K 8/375; A61K 8/42; A61K 2800/412; A61K 2800/26; A61K 2800/5424; A61K 2800/30

(57) ABSTRACT

The present invention relates to a sulfate-free aqueous concentrate comprising at least: (i) at least 25% by weight of opacifier particles selected from C16-C22 fatty acid, C16-C22 fatty alcohol, mono-, di- and tri-C16-C22 esters of glycerol, mono- and di-C16-C22 esters of ethylene glycol, diethylene glycol and triethylene glycol, C16-C22 fatty acid alkanolamides and mixtures thereof, with said particles are having a median diameter ranging from 1 to 15 μm; (ii) an amphoteric or zwitterionic surfactant, (iii) water, with the total amount of surfactants in said aqueous concentrate ranging from 0.5 to 15% by weight, relative to the total weight of the aqueous concentrate. It also relates to the use of such an aqueous concentrate as an opacifier. The aqueous concentrate of the invention may be used as an opacifier in a composition, to replace at least some of a styrene opacifier or titanium dioxide in said composition.

This application claims priorities to U.S. PA No. 62/769,757 filed on Nov. 20, 2018 and to U.S. PA No. 62/827,919, filed on Apr. 2, 2019, the whole content of each of these applications being incorporated herein by reference for all purposes.

The present invention relates to opacifier concentrates, more particularly to opacifier concentrates for use in aqueous compositions.

Additives are commonly used to modify the appearance of aqueous compositions. The appearance modification imparted by such additives is influenced by the opacity and shine exhibited by the relevant additive and may range from a highly pearlescence appearance, that is, an iridescent opacity, wherein the additive exhibits a high pearl shine, to no pearlescence, that is, a dull or matte opacity, wherein the additive exhibits no pearl shine. In the latter case, the additive may advantageously provide a white opaque visual aspect (milky appearance).

The present invention relates more particularly to opacifier additives mainly imparting a dull or matte opacity (seen by naked eye) to personal care formulations containing them.

It is already known to add such opacifier additives into personal care products that are initially transparent or translucent. This is because white opaque visual appearance is often associated with the impression that the product has a luxurious or smooth texture, a rich feel, a nutritious or healthy formula and/or a moisturizing effect.

One of the most widely used opacifier additive today in personal care compositions is styrene polymers, such as styrene acrylates and styrene acrylamides.

There is however an overwhelming trend towards alternative opacifier additives, in particular towards zero-plastic products in order to replace such petroleum-derived synthetic opacifier additives of the prior art.

Ethylene glycol distearate-based waxy opacifiers providing both opacity and pearl shine have already been disclosed in the prior art.

There remain however a need for improved opacifier particles that are suitable or intended for use in aqueous compositions and that are more environmentally-friendly compared to styrene polymers or titanium dioxide, in particular for opacifier particles that are not petroleum-derived.

There remain also a need for improved not petroleum-derived opacifier particles that provide greater opacity and whiteness, compared to opacifier particles already known from the prior art.

It is also desirable to provide opacifier particles yielding to aqueous composition with improved stability over time.

As a matter of fact, some of the opacifiers of the prior art are not stable at particular pHs or in combination with particular substances typically present in some personal care products.

It is also desirable to provide opacifier particles that may be readily added into aqueous compositions, and in particular to opacifier particles that can be implemented without any constraints as regards existing manufacturing processes.

Some of the opacifiers of the prior art, such as for instance TiO₂ or kaolin-based opacifiers, are not totally satisfactory today.

This is the reason why it may be desirable to provide opacifier particles that overcome at least one, or more, of the above-mentioned problems.

It is particularly desirable to provide opacifier particles in the form of an aqueous concentrate. As a matter of fact, use of a concentrate allows increased consistency and allows for a reduction in the manufacturing time required for making the finished formulation.

Surprisingly it has been found that opacifier concentrates as defined below provide improved opacity and whiteness, without negative impacts for instance regarding stability over time and/or ease of use.

The present invention relates to a sulfate-free aqueous concentrate comprising at least:

i) at least 25% by weight of opacifier particles selected from C16-C22 fatty acid, C16-C22 fatty alcohol, mono-, di- and tri-C16-C22 esters of glycerol, mono- and di-C16-C22 esters of ethylene glycol, diethylene glycol and triethylene glycol, C16-C22 fatty acid alkanolamides and mixtures thereof, with said particles are having a median diameter ranging from 1 to 15 μm;

ii) an amphoteric or zwitterionic surfactant,

iii) water, with the total amount of surfactants in said aqueous concentrate ranging from 0.5 to 15% by weight, relative to the total weight of the aqueous concentrate.

The present invention also relates to the use of such an aqueous concentrate as an opacifier.

According to anyone of the invention embodiments, the aqueous concentrate of the invention may be used as an opacifier in a composition, to replace at least some of a styrene opacifier or titanium dioxide in said composition.

Certain embodiments of the present invention may provide one or more of the following advantages compared, for example, to opacifiers currently used in home or personal care compositions:
Environmentally friendly opacifier;
Less skin irritation and/or allergy;
Improved stability at low and high pHs (e.g. less than pH 5.5 and pH between 7 and 9 respectively);
Good stability in the presence of cationic polymers (e.g. cationic conditioning agents);
Good stability at high electrolyte concentrations;
Good stability in the presence of ionic substances (e.g. anionic surfactants);
Whiteness, yellowness and/or opacity that is comparable to typically used opacifiers or improved;
No sandy feel;
Minimal agglomeration;
Minimal change in viscosity of the aqueous composition;
Good tolerance on skin, minimal ocular irritation;
Minimal microorganism contamination;
Improved colour intensity, when combined with colouring agents into coloured composition

The present invention also relates to a method for modifying the appearance of a composition, comprising adding an aqueous concentrate as defined previously to such composition.

The present invention also relates to a method to increase the opacity and/or whiteness of a composition, comprising adding an aqueous concentrate as defined previously to such composition.

In some embodiments, advantageous effects can be obtained using amounts of opacifier particles that are comparable to the amount of another typical opacifier that would be used to obtain the same effect.

Advantageously, especially when the aqueous concentrate of the invention further contains a surfactant, the opacifier particles of the invention may be readily added into aqueous compositions without any constraints as regards existing manufacturing processes, providing a high flexibility.

For instance, the aqueous concentrate of the invention may be easily added into the aqueous composition any time during its manufacturing process, such as for example “on top”, namely directly “as is” at the end of the manufacturing process.

The aqueous concentrate of the invention is provided in a liquid form that can be readily used: there is no need to make any premix formulation beforehand. For the avoidance of any doubt the amounts of component (such as opacifier particles or surfactants) in an aqueous concentrate of the invention refer to the actual amount of active material present in the concentrate.

Should the disclosure of any patents, patent applications, and publications which are incorporated herein by reference conflict with the description of the present application to the extent that it may render a term unclear, the present description shall take precedence.

Opacifier Particles

The aqueous concentrate of the invention comprises opacifier particles in order to provide a milky appearance to the composition into which said aqueous concentrate is to be added.

The aqueous concentrate of the invention comprises opacifier particles selected from C16-C22 fatty acid, C16-C22 fatty alcohol, mono-, di- and tri-C16-C22 esters of glycerol, mono- and di-C16-C22 esters of ethylene glycol, diethylene glycol and triethylene glycol, C16-C22 fatty acid alkanolamides and mixtures thereof.

According to anyone of the invention embodiments, the opacifier particles may be mono- or di-C16-C22 esters of ethylene glycol and mixtures thereof. According to anyone of the invention embodiments, the opacifier particles may comprise essentially di-C16-C22 esters of ethylene glycol.

In one embodiment, the aqueous concentrate of the invention may be substantially free of mono-C16-C22 esters of ethylene glycol, i.e. it may comprise from 0 to less than 1% by weight of mono-C16-C22 esters of ethylene glycol, for instance from 0 to less than 0.8% by weight, for instance from 0 to less than 0.5% by weight, relative to the total weight of the aqueous concentrate. An aqueous concentrate of the invention may even comprise no mono-C16-C22 esters of ethylene glycol, i.e. 0% by weight of mono-C16-C22 esters of ethylene glycol per 100% by weight of the aqueous concentrate.

According to another invention embodiments, the opacifier particles may be especially a mixture of mono-C16-C22 esters of ethylene glycol and of di-C16-C22 esters of ethylene glycol, for instance in a ratio [mono-C16-C22 esters of ethylene glycol:di-C16-C22 esters of ethylene glycol] ranging from 1:10 to 10:1, for instance from 1:5 to 5:1, for instance from 1:4 to 4:1, for instance from 1:3 to 1:4.

According to anyone of the invention embodiments, the opacifier particles may also comprise stearic acid.

For instance, when the opacifier particles comprise stearic acid in addition to a mixture of mono-C16-C22 esters of ethylene glycol and of di-C16-C22 esters of ethylene glycol as described previously, stearic acid may be present in an amount ranging from 5 to 20% by weight, relative to the total weight of said mixture of mono-C16-C22 esters of ethylene glycol and of di-C16-C22 esters of ethylene glycol.

According to anyone of the invention embodiments, the opacifier particles may be ethylene glycol distearate.

The appearance imparted by such particles is believed to arise from their crystal morphology.

According to anyone of the invention embodiments, the crystal shape of the opacifier particles used in a concentrate of the invention is near-spherical. According to anyone of the invention embodiments, the opacifier particles may have a median diameter lower than 14 μm, for instance lower than 12 μm.

According to anyone of the invention embodiments, the opacifier particles may have a median diameter greater than 2 μm, for instance greater than 4 μm. According to anyone of the invention embodiments, the opacifier particles may have a median diameter ranging from 3 to 8 μm.

According to anyone of the invention embodiments, the opacifier particles may have a median diameter ranging from 4 to 6 μm.

Unless otherwise stated, the particle sizes referred to herein are measured in a well-known manner by laser diffraction.

The central idea in laser diffraction is that a particle will scatter light at an angle determined by that particle's size. Larger particles will scatter at small angles and smaller particles scatter at wide angles. A collection of particles will produce a pattern of scattered light defined by intensity and angle that can be transformed into a particle size distribution result.

According to anyone of the invention embodiments, particle sizes may be measured using laser diffraction granulometer Partica LA-960 as supplied by HORIBA. HORIBA LA-960 particle size analyzer uses the laser diffraction method to measure size distributions. This technique uses first principles to calculate size using light scattered off the particle (edge diffraction) and through the particle (secondary scattering refraction). HORIBA LA-960 particle size analyzer then provides a value corresponding to the median diameter.

According to anyone of the invention embodiments, the opacifier particles may have a melting point greater than 50° C.

Melting point may be measured for example using a TA Instruments Q200 Differential Scanning calorimeter.

The following operating conditions may be for instance implemented for a sample of 20 milligram of product placed in an aluminum Tzero pans: 3° C./min, with a scan rate from 30° C. to 75° C. single pass scan

According to anyone of the invention embodiments, the aqueous concentrate of the invention may contain at least 20% by weight, for example at least 25% by weight, for example at least 30% by weight, of said opacifier particles, relative to the total weight of the aqueous concentrate.

According to anyone of the invention embodiments, the aqueous concentrate of the invention may contain up to 75% by weight, for example up to 70% by weight, for example up to 60% by weight, for example up to 50% by weight of said opacifier particles, relative to the total weight of the aqueous concentrate. According to anyone of the invention embodiments, the aqueous concentrate of the invention may contain from 20 to 75% by weight, for example from 20 to 60% by weight, for example from 20 to 50% by weight, for example from 30 to 50% by weight of said opacifier particles, relative to the total weight of the aqueous concentrate.

According to anyone of the invention embodiments, the aqueous concentrate of the invention may contain from 25 to 75% by weight, for example from 25 to 60% by weight, for example from 25 to 50% by weight, of said opacifier particles, relative to the total weight of the aqueous concentrate.

According to a preferred invention embodiments, an aqueous concentrate of the invention contains at least 30% by weight of said opacifier particles, relative to the total weight of the aqueous concentrate, for instance at least 31% by weight. Preferably, the aqueous concentrate of the invention may contain at least 30% by weight, for example from 30 to 75% by weight, for example from 30 to 60% by weight, for example from 30 to 50% by weight of said opacifier particles, relative to the total weight of the aqueous concentrate. It has been found that it was advantageously possible to achieve very high loads of opacifier particles in an aqueous concentrate of the invention, and especially loads greater than 30% by weight, relative to the total weight of the concentrate, without negative impacts for instance regarding stability over time and/or ease of use.

There is a continuing need for high load concentrates, and achieving higher loads in opacifier particles is extremely valuable in particular for economic reasons (in particular for reducing the weight of the compositions and, consequently, the transportation costs thereof), the concentrated formulation then being diluted to the desired concentration by the final user.

The aqueous concentrate of the invention is intended to be added into a composition in order to modify its appearance, and in particular in order to increase its opacity and/or whiteness.

The amount of aqueous concentrate to be added into a composition depends on the target opacity.

As mentioned previously, the present invention also relates to a method for modifying the appearance of a composition, comprising adding an aqueous concentrate of the invention to such composition.

The present invention also relates to a method to increase the opacity and/or whiteness of a composition, comprising adding an aqueous concentrate of the invention to such composition.

According to anyone of the invention embodiments, the aqueous concentrate of the invention may be added in an amount of at least 1% by weight, for example of at least 1.5% by weight, relative to the total weight of the composition. According to anyone of the invention embodiments, the aqueous concentrate of the invention may be added in an amount ranging from 1 to 5% by weight, for example from 1.5 to 5% by weight, for example from 1.5 to 4.5% by weight, relative to the total weight of the composition.

Whiteness

According to anyone of the invention embodiments, the aqueous concentrate may, for example, have a L* whiteness of at least about 80.

For example, the aqueous concentrate of the invention may have a L* whiteness equal to or greater than about 82 or equal to or greater than about 84 or greater to or greater than about 86 or equal to or greater than about 88 or equal to or greater than about 90.

The aqueous concentrate of the invention may, for example, have a L* whiteness up to about 100 or up to about 99 or up to about 98 or up to about 97 or up to about 96.

The aqueous concentrate of the invention may, for example, have a L* whiteness ranging from about 84 to about 98.

In one of the invention embodiments, the aqueous concentrate of the invention may, for example, have a L* whiteness ranging for example from about 86 to about 96, for example from about 88 to about 94.

In another one of the invention embodiments, the aqueous concentrate of the invention may, for example, have a L* whiteness ranging for example from about 86 to about 98, for example from about 88 to about 98.

According to anyone of the invention embodiments, a composition containing an aqueous concentrate of the invention may, for example, have a L* whiteness of at least about 80.

For example, the compositions may have a L* whiteness equal to or greater than about 82 or equal to or greater than about 84 equal to or greater than about 85 or equal to or greater than about 86 or equal to or greater than about 88 or equal to or greater than about 90 or equal to or greater than about 92 or equal to or greater than about 94 or equal to or greater than about 95.

The compositions may, for example, have a L* whiteness up to about 100 or up to about 99 or up to about 98 or up to about 97 or up to about 96.

The compositions may, for example, have a L* whiteness ranging from about 80 to about 90 or from about 80 to about 85.

The L* whiteness is determined using the L*a*b* colour space. L* whiteness of a composition may be measured using the spectrocolorimeter DR LANGE LUCI 100 (measuring geometry d/8° acc. to DIN 5033).

The plates that are used to measure L* whiteness consist of hexagonal shaped cells 2 cm wide by 3 mm thick. The sample quantity for each measurement is 1.5 mL per cell.

Covering Effect

According to anyone of the invention embodiments, the aqueous concentrate may, for example, exhibit a covering effect of at least about 90%.

For example, the aqueous concentrate of the invention may have a covering effect equal to or greater than about 92% or equal to or greater than about 94% or greater to or greater than about 96% or equal to or greater than about 98% or equal to.

The aqueous concentrate of the invention may, for example, have a covering effect up to about 100% or up to about 99% or up to about 98%.

The aqueous concentrate of the invention may, for example, have a covering effect ranging from about 92% to about 99%, for example from about 94% to about 99%, for example from about 96% to about 98%.

According to anyone of the invention embodiments, a composition containing an aqueous concentrate of the invention may, for example, have a covering effect of at least about 50%.

For example, the compositions may have a covering effect equal to or greater than about 55% or equal to or greater than about 60% or equal to or greater than about 65% or equal to or greater than about 70% or equal to.

The compositions may, for example, have a covering effect up to about 100% or up to about 98%% or up to about 95% or up to about 90%.

The compositions may, for example, have a covering effect ranging from about 55% to about 98% or from about 60% to about 95%.

Covering effect may be measured by the contrast method, using the spectrocolorimeter DR LANGE LUCI 100 (measuring geometry d/8° acc. To DIN 5033).

Covering effect of a composition is defined as being the ratio between L* whiteness of said composition when placed on a black background (L*_{black background}) and L* whiteness of said composition when placed on a white background (L*_{white background}).

Covering effect(expressed as a%)=(L*_{black background})/(L*_{white background})×100

The plates that are used to measure L* whiteness on each background (black or white) consist of-hexagonal shaped cells 2 cm wide by 3 mm thick. The sample quantity for each measurement is 1.5 mL per cell.

Color Intensity

In addition to opacifier additives, it is common practice to include colouring agents, including dyes and pigments, into compositions in order to improve their aesthetic appearance.

The term “pigments” should be understood as meaning mineral or organic, coloured particles, which are insoluble in the hydrophilic or lipophilic liquid phase and which are intended to colour the composition including them. Colouring agent are generally present in a concentration that is sufficient to afford the desired colour.

Combinations with conventional opacifier additives, such as styrene polymers, may have the effect of altering the color shade and/or of producing a grayish or even ashen effect, which for obvious aesthetic reasons is particularly undesirable.

It is therefore desirable to provide opacifier particles that do not significantly alter the color shade of aqueous coloured composition, and more especially that do not produce a grayish or even ashen effect.

It is also desirable to provide opacifier particles that improve color intensity of aqueous coloured composition,

Surprisingly it has been found that opacifier concentrates of the invention provide improved color intensity when added into coloured compositions further containing a colouring agent, without negative impacts for instance regarding color shade and/or without producing greying or blueing effect. According to anyone of the invention embodiments, a composition containing an aqueous concentrate of the invention may further contain a colouring agent, which may be liposoluble or water-soluble.

The colouring agents may be synthetic or natural.

They may be organic or mineral dyes.

The natural or synthetic liposoluble dyes are, for example, DC Red 17, DC Red 21, DC Red 27, DC Green 6, DC Yellow 11, DC Violet 2, DC Orange 5, Sudan red, carotenes ([beta]-carotene or lycopene), xanthophylls (capsanthin, capsorubin or lutein), palm oil, Sudan brown, quinoline yellow, annatto and curcumin.

The natural or synthetic water-soluble dyes are, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper-containing chlorophylline, methylene blue, anthocyanins (enocyanin, black carrot, hibiscus or elder), caramel and riboflavin.

The dyes may also be chosen from juglone, lawsone, extracts of fermented soya, of algae, of fungi or of microorganisms, flavylium salts not substituted in position 3, extracts of Gesneria fulgens, Blechum procerum or Saxifraga and pigments that may be obtained by extraction with an organic or aqueous-organic solvent of a culture medium of micromycetes of the Monascus type. The composition may also comprise pigments in dispersed form.

Non-limiting illustrations of mineral pigments that may be mentioned more particularly include yellow, red or brown metal oxides, for instance iron oxides.

As metal powders, mention may be made of copper powder.

As illustrations of pigments that are more particularly suitable for the invention, mention may be made especially of brown iron oxide and yellow iron oxide, coated with perfluoroalkyl phosphate, and alumina-treated titanium oxide, coated with perfluoroalkyl phosphate, in particular such as the pigmentary pastes sold under the trade names Yellow Iron Oxide Covafluor, PF5 Yellow 601 (yellow) and PF5 R516L (red) by the company Daito, and under the trade names FA50DRF, FA50DYF, FA65DF and FA65 DBF by the company Kobo.

The composition may also comprise nacres in dispersed form, with the proviso that they do not affect the expected properties of the composition.

The term “nacres” should be understood as meaning coloured particles of any form, which may or may not be iridescent, especially produced by certain molluscs in their shell, or alternatively synthesized, and which have a colour effect via optical interference.

The nacres may be chosen from nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

As illustrations of nacres that may be used in the context of the present invention, mention may be made in particular of gold-coloured nacres sold especially by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the names Bronze fine (17384) (Colorona) and Bronze (17353) (Colorona) and by the company Engelhard under the name Super bronze (Cloisonne); the orange nacres sold especially by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold especially by the company Engelhard under the names Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red-tinted nacres with a golden tint sold especially by the company Engelhard under the name Sunstone G012 (Gemtone); the pink nacres sold especially by the company Engelhard under the name Tan opale G005 (Gemtone); the black nacres with a golden tint sold especially by the company Engelhard under the name Nu antique bronze 240 AB (Timica); the blue nacres sold especially by the company Merck under the name Matte blue (17433) (Microna); the white nacres with a silvery tint sold especially by the company Merck under the name Xirona Silver; and the golden-green pinkish-orange nacres sold especially by the company Merck under the name Indian summer (Xirona), and mixtures thereof.

According to a specific embodiment, a composition according to the invention further contains at least one colouring agent, preferably at least one water-soluble colouring agent, preferably at least one water-soluble dye.

According to a specific embodiment, a composition according to the invention contain less than 2%, in particular less than 1%, more particularly less than 0.5% by weight, for instance 0% by weight of nacres, relative to their total weight of nacres.

Surfactant

The aqueous concentrate of the invention further comprises a surfactant. According to anyone of the invention embodiments, the aqueous concentrate of the invention comprises an amphoteric or zwitterionic surfactant.

Typical examples of amphoteric or zwitterionic surfactants are alkyl betaines, alkylamidopropylbetaines, amphoacetate, amphopropionate, aminopropionates, aminoglycinates, imidazolinium betaines, sulfobetaines and amine oxide Alkylamidobetaines and especially cocamidopropyl betaine are preferred for the purposes of the invention.

According to anyone of the invention embodiments, the aqueous concentrate of the invention may further comprise, in addition to amphoteric or zwitterionic surfactants a non ionic surfactant.

Typical examples of non ionic surfactants are:
alkyl polyglucosides of the general formula: R¹—O—(R²OₙO—Zₓ
wherein R¹ is an alkyl group with 8 to 18 carbon atoms, R² is an ethylene or propylene group, Z is a saccharide group with 5 to 6 carbon atoms, n is a number from 0 to 10 and x is a number between 1 and 5.
Examples are decyl glucoside, carpylyl glucoside, ceteary glucoside, cocoyl ethyl glucoside, lauryl glucoside, myristyl glucoside and coco glucoside;
C₁₀-C₂₂-fatty alcohol ethoxylates, such as C₁₀-C₂₂-fatty alcohol ethers with the average degree of ethoxylation between 1 and 25, preferably 2 and 20, more preferably 2 to 10.
Suitable examples are oleth-2, oleth-3, oleth-4, oleth-5, oleth-6, oleth-7, oleth-8, oleth-9, oleth-10, oleth-11, oleth-12, oleth-15, oleth-16, oleth-20, oleth-25, laureth-2, laureth-3, laureth-4, laureth-5, laureth-6, laureth-7, laureth-8, laureth-9, laureth-10, laureth-11, laureth-12, laureth-13, laureth-15, laureth-16, laureth-20, laureth-25, ceteth-10, ceteth-12, ceteth-14, ceteth-15, ceteth-16, ceteth-17, ceteth-20, ceteth-25, cetoleth-10, cetoleth-12, cetoleth-14, cetoleth-15, cetoleth-16, cetoleth-17, cetoleth-20, cetoleth-25, ceteareth-10, ceteareth-12, ceteareth-14, ceteareth-15, ceteareth-16, ceteareth-18, ceteareth-20, ceteareth-22, ceteareth-25, isosteareth-10, isosteareth-12, isosteareth-15, isosteareth-20, isosteareth-22, isosteareth-25, steareth-10, steareth-11, steareth-14, steareth-15, steareth-16, steareth-20, and steareth-25.

According to anyone of the invention embodiments, an aqueous concentrate according to the invention may contain less than 50% by weight, for example from 5 to 40% by weight, for example from 10 to 30% by weight, for example from 10 to 25% by weight of amphoteric or zwitterionic surfactants and/or of non ionic surfactants, relative to the total weight of the aqueous concentrate.

According to a preferred invention embodiments, an aqueous concentrate according to the invention contains less than 15% by weight, for example from 0.1 to 15% by weight, for example from 0.5 to 15% by weight, for example from 1 to 10% by weight of surfactant, relative to the total weight of the aqueous concentrate.

According to a preferred invention embodiments, the total amount of surfactants in an aqueous concentrate according to the invention may range from 0.5 to 15% by weight, especially from 1 to 10% by weight, for instance from 4 to 8% by weight, relative to the total weight of the aqueous concentrate.

According to a preferred invention embodiments, an aqueous concentrate according to the invention may contain, as surfactant, only amphoteric or zwitterionic surfactants as described previously.

According to a preferred invention embodiments, an aqueous concentrate according to the invention may contain amphoteric or zwitterionic surfactants as described previously in an amount ranging from 0.5 to 15% by weight, especially from 1 to 10% by weight, for instance from 4 to 8% by weight, relative to the total weight of the aqueous concentrate.

It has been found unexpectedly that the aqueous concentrate according to the invention was able to provide improved opacity and whiteness, without negative impacts for instance regarding stability over time and/or ease of use, even when such lower amounts of surfactants, especially amphoteric or zwitterionic surfactants, were present.

In particular, it has been found that it was possible to prepare improved aqueous concentrate including very high loads of opacifier particles (and especially containing at least 30% by weight of opacifier particles relative to the total weight of the aqueous concentrate) when using amphoteric or zwitterionic surfactants as described previously, in amounts ranging from 0.5 to 15% by weight, especially from 1 to 10% by weight, for instance from 2 to 10% by weight, for instance from 4 to 8% by weight, relative to the total weight of the aqueous concentrate, without the need of any additional surfactant, and especially without the need for additional non-ionic and/or anionic surfactants. The aqueous concentrate according to the invention is a sulfate-containing surfactants free concentrate.

By the expression “sulfate-containing surfactants free concentrate” or “sulfate-free concentrate” it is meant that the concentrate of the invention is devoid of, i.e. does not contain (0%) any anionic surfactant which is a derivative of a sulfate, such as especially sodium lauryl sulfate (SLS), sodium laureth sulfate (SLES), ammonium lauryl sulfate (ALS) or ammonium laureth sulfate (ALES). For the purposes of the present invention, the term “anionic surfactant which is a derivative of a sulfate” means surfactants comprising at least one anionic group or group that can be ionized into an anionic group, chosen from sulfate functions (—OSO3H or —OSO3-).

Thus, the following anionic surfactants are not present in the concentrates according to the invention: salts of alkyl sulfates, of alkylamide sulfates, of alkyl ether sulfates, of alkylamido ether sulfates, of alkylaryl ether sulfates, of monoglyceride sulfates.

According to a preferred invention embodiments, an aqueous concentrate according to the invention may contain from 0 to less than 2% by weight of non ionic surfactant, such as for instance cocoglucoside, relative to the total weight of the aqueous concentrate.

More typically an aqueous concentrate according to the invention may comprise, based on 100% by weight of such concentrate, from 0 to less than 1% by weight of non ionic surfactant and even substantially no non ionic surfactant, i.e. from 0 to less than 0.1% by weight of non ionic surfactant per 100% by weight of the concentrate, more typically no non ionic surfactant, i.e. 0% by weight non ionic surfactant per 100% by weight of the concentrate.

According to a preferred invention embodiments, an aqueous concentrate according to the invention may contain from 0 to less than 2% by weight of anionic surfactant, relative to the total weight of the aqueous concentrate.

More typically an aqueous concentrate according to the invention may comprise, based on 100% by weight of such concentrate, from 0 to less than 1% by weight of anionic surfactant and even substantially no anionic surfactant, i.e. from 0 to less than 0.1% by weight of anionic surfactant per 100% by weight of the concentrate, more typically no anionic surfactant, i.e. 0% by weight anionic surfactant per 100% by weight of the concentrate.

According to a preferred invention embodiments, an aqueous concentrate according to the invention may contain from 0 to less than 2% by weight of emollient, such as for instance glyceryl oleate, relative to the total weight of the aqueous concentrate.

More typically an aqueous concentrate according to the invention may comprise, based on 100% by weight of such concentrate, from 0 to less than 1% by weight of emollient and even substantially no emollient, i.e. from 0 to less than 0.1% by weight of emollient per 100% by weight of the concentrate, more typically no emollient, i.e. 0% by weight emollient per 100% by weight of the concentrate. According to a preferred invention embodiments, an aqueous concentrate of the invention is characterized in that:
it contains at least 30% by weight of said opacifier particles, relative to the total weight of the aqueous concentrate, for instance at least 31% by weight,
it contains amphoteric or zwitterionic surfactants in an amount ranging from 0.5 to 15% by weight, especially from 1 to 10% by weight, for instance from 4 to 8% by weight, relative to the total weight of the aqueous concentrate,
it contains from 0 to less than 2% by weight of non ionic surfactant, such as for instance cocoglucoside, relative to the total weight of the aqueous concentrate,
it contains from 0 to less than 2% by weight of anionic surfactant, such as for instance sodium lauryl sulfate (SLS) or sodium laureth sulfate (SLES), relative to the total weight of the aqueous concentrate, and/or
   it contains from 0 to less than 2% by weight of emollient, such as for instance glyceryl oleate, relative to the total weight of the aqueous concentrate.

According to anyone of the invention embodiments, an aqueous concentrate according to the invention may contain from 0 to less than 2% by weight of alkyl silicone, namely modified silicone with fatty alkyl chains grafted to the silicone backbone such as for instance dimethiconol stearate and dimethiconol behenate, relative to the total weight of the aqueous concentrate.

More typically an aqueous concentrate according to the invention may comprise, based on 100% by weight of such concentrate, from 0 to less than 1% by weight of alkyl silicone and even substantially no alkyl silicone, i.e. from 0 to less than 0.1% by weight of alkyl silicone per 100% by weight of the concentrate, more typically no alkyl silicone, i.e. 0% by weight alkyl silicone per 100% by weight of the concentrate.

According to anyone of the invention embodiments, an aqueous concentrate according to the invention may contain from 0 to less than 2% by weight of ethoxylated glycerides derived from carboxylic acids having 6 to 22 carbon atoms, such as for instance ethoxylated glycerides with an average ethoxylation degree of 2 ethylene oxide mol per glycerine mol and having acyl groups deriving from coconut oil, relative to the total weight of the aqueous concentrate. More typically an aqueous concentrate according to the invention may comprise, based on 100% by weight of such concentrate, from 0 to less than 1% by weight of ethoxylated glycerides derived from carboxylic acids having 6 to 22 carbon atoms and even substantially no ethoxylated glycerides derived from carboxylic acids having 6 to 22 carbon atoms, i.e. from 0 to less than 0.1% by weight of ethoxylated glycerides derived from carboxylic acids having 6 to 22 carbon atoms per 100% by weight of the concentrate, more typically no ethoxylated glycerides derived from carboxylic acids having 6 to 22 carbon atoms, i.e. 0% by weight ethoxylated glycerides derived from carboxylic acids having 6 to 22 carbon atoms per 100% by weight of the concentrate.

Water

The concentrate of the invention contains water.

According to anyone of the invention embodiments, an aqueous concentrate of the invention may contain at least 20% by weight, for example from 20 to 60% by weight, for example at least 30% by weight, for example at least 40% by weight, for example from 40 to 60% by weight of water, relative to the total weight of the aqueous concentrate.

According to anyone of the invention embodiments, an aqueous concentrate of the invention is substantially free of glycerol monostearate, i.e. it comprises from 0 to less than 1% by weight of glycerol monostearate, for instance from 0 to less than 0.8% by weight, for instance from 0 to less than 0.5% by weight, relative to the total weight of the aqueous concentrate.

An aqueous concentrate of the invention may even comprise no glycerol monostearate, i.e. 0% by weight of glycerol monostearate per 100% by weight of the aqueous concentrate.

Solids Content

According to anyone of the invention embodiments, an aqueous concentrate of the invention may have a solids content of at least 20% by weight and up to 80% by weight, for example up to 90% by weight, or even up to 100% by weight. An aqueous concentrate of the invention may have for example a solids content from 25 to 60% by weight, for example of at least 30% by weight, for example of at least 40% by weight.

Solids content may be measured for instance on a Mettler Toledo HG63 Moisture Balance blank.

The following operating conditions may be for instance implemented for a sample of one gram of product placed in an aluminum capsule: said sample may be placed at a temperature of 110° C. for 75 minutes.

Composition

The aqueous concentrate of the invention can be useful for modifying the appearance and/or increasing the opacity and/or whiteness of a a wide variety of aqueous compositions such as personal care, health care, home care, and institutional and industrial care compositions.

The term “personal care composition” as used herein means compositions, including but not limited to cosmetics, toiletries, cosmeceuticals, beauty aids, personal hygiene and cleansing compositions for application to the body, including the skin, hair, scalp, and nails, of humans and animals. Typical examples of personal care compositions include for instance liquid soaps (e.g. hand soaps, body washes, hair shampoos), hair conditioners, hair styling products, sun lotions, moisturizers, deodorants or make-up compositions (e.g. lipsticks).

The term “health care compositions” as used herein means compositions including but not limited to pharmaceuticals, pharmacosmetics, oral (mouth and teeth) care compositions, such as oral suspensions, mouthwashes, toothpastes, and the like, and over-the-counter compositions for external application to the body, including the skin, scalp, nails, and mucous membranes of humans and animals, for ameliorating a health-related or medical condition, or for generally maintaining hygiene or well-being.

The term “home care compositions” as used herein means compositions including, but not limited to, compositions for use in a domestic household for surface cleaning or maintaining sanitary conditions, such as in the kitchen and bathroom, and laundry products for fabric care and cleaning, and the like. The term “institutional and industrial care compositions” as used herein means compositions, including but not limited to, cleaning compositions, for use in surface cleaning or maintaining sanitary conditions in institutional and industrial environments, and compositions for treating textiles.

According to anyone of the invention embodiments, the aqueous concentrate of the invention may be added into compositions without dramatically impacting its viscosity.

The compositions in which the aqueous concentrate of the invention is to be added would generally have an apparent viscosity comprised between 1,500 and 50,000 cps, for instance comprised between 2,000 and 30,000 cps, for instance comprised between 2,000 and 25,000 cps, for instance comprised between 2,500 and 20,000 cps. The apparent viscosity of a composition may be measured after 24-hours in a temperature-controlled room (21±3° C.), using a Brookfield Viscosimeter Model DV-II at 10 RPM, with a RV spindle 4. According to one embodiment, the composition containing an aqueous concentrate of the invention may have an apparent viscosity greater than 1,500 cps, for instance greater than 2,000 cps, for instance greater than 3,000 cps.

According to anyone of the invention embodiments, the aqueous concentrate of the invention may further comprise preservatives and/or pH adjusting agents.

The concentrate of the present invention may optionally comprise, based on the total weight of the concentrate, a total amount of up to about 5% by weight, for example from about 0.1% by weight to 5% by weight, of preservatives and/or pH adjusting agents.

According to anyone of the invention embodiments, the pH of the aqueous concentrate of the invention may typically range between 4 and 6, for instance between 4 and 5.

Optional components can also be utilized in the concentrates of the present invention as a convenient means of incorporation into personal care, home care, health care, and I&I care compositions. Such conventional optional ingredients are well known to those skilled in the art, e. g. thickeners and viscosity modifiers; dyes; electrolytes; and sequestering agents as well as the numerous other optional components for enhancing and maintaining the properties of a desired personal care, home care, health care, and I&I care composition.

Such agents may each independently be present at levels of up to about 5% by weight, more typically from about 0.01% by weight to about 2.0% by weight, relative to the total weight of the concentrate.

### EXAMPLES

### Example 1: Preparation of Aqueous Concentrates in Accordance with the Invention

### Example 1a: Preparation of Aqueous Concentrate A

An aqueous concentrate of the invention (Aqueous concentrate A) has been prepared according to the following procedure.

34.5 wt % of Ethylene glycol distearate (Mackester GDSV flakes, available from Solvay), 34.5 wt % of water, and 7.7 wt % of Cocamidopropyl Betaine (Mackam 35, available from Solvay) are added together into a grinding pot.

This mixture is mixed with a high shear Cowles blade at 700 RPM until the coarse slurry is transformed into a smooth flowing fluid with viscosity between 350 and 450 cP. Cooling is applied to the grinding pot to maintain the temperature below 15° C., under stirring with an agitator.

The slurry is then pumped through an horizontal mill (temperature maintained below 35° C. exiting the mill).

14.2 wt % of Cocamidopropyl Betaine (Mackam 35, available from Solvay) and 8.4 wt % of water are added to the milled concentrate. The pH is adjusted to about 4.8 by adding Citric Acid. The solids level is measured using a moisture balance to determine if additional water is needed to reach a final solids content of about 46%.

Aqueous concentrate A is a white liquid at room temperature that does not exhibit pearl shine.

It has a melting point, measured according to the protocole of the description, greater than 50° C.

### Example 1b: Preparation of Aqueous Concentrate B

Another aqueous concentrate of the invention (Aqueous concentrate B) has been prepared according to the following procedure.

45 wt % of Ethylene glycol distearate (Mackester GDSV flakes, available from Solvay), 45 wt % of water, and 10 wt % of Cocamidopropyl Betaine (Mackam CAB 818, available from Solvay) are added together into a grinding pot.

This mixture is mixed with a high shear Cowles blade at 700 RPM until the coarse slurry is transformed into a smooth flowing fluid with viscosity between 350 and 450 cP. Cooling is applied to the grinding pot to maintain the temperature below 15° C., under stirring with an agitator.

The slurry is then pumped through an horizontal mill (temperature maintained below 35° C. exiting the mill).

14.2 wt % of Cocamidopropyl Betaine (Mackam CAB 818, available from Solvay) and 8.4 wt % of water and 0.5% of sodium Benzoate are added to the milled concentrate. The pH is adjusted to about 4.8 by adding Citric Acid. The solids level is measured using a moisture balance to determine if additional water is needed to reach a final solids content of about 42%.

Aqueous concentrate B is a white liquid at room temperature that does not exhibit pearl shine.

It has a melting point, measured according to the protocole of the description, greater than 50° C.

### Example 1c: Stability Upon Storage of Aqueous Concentrate A and Aqueous Concentrate B of the Invention

Stability upon storage of aqueous concentrates A and B has also been tested according to the following temperature storage conditions:

“4° C.” means storage of the aqueous concentrate at 4° C. in a Memert ICP 450 ventilated oven.

“40° C.” means storage of the aqueous concentrate at 40° C. in a Memert IC 450 ventilated oven.

“Freeze/thaw” means submitting the aqueous concentrate to 5 freeze and thaw cycles, each cycle being composed by the following steps: decreasing the temperature from 25° C. to −9° C. in 8 hours; plateaus at −9° C. during 8 hours; increasing the temperature to reach 25° C. during 8 hours; and then plateaus at 25° C. during 8 hours.

For the “4° C.” and “40° C.” temperature storage test, viscosity is measured weekly on a Brookfield DVII+RV viscometer with a spindle rotation speed of 10 rpm.

For the “freeze/thaw” test, viscosity is measured at the end of the five cycles on a Brookfield DVII+RV viscometer with a spindle rotation speed of 10 rpm.

In each test, the appearance of the sample and its particle size (Horiba granulometer) are also assessed.

It has been established that aqueous concentrates A and B are stable at least 3 months in such temperature storage conditions (4° C., 40° C.).

It has been established that aqueous concentrates A and B are stable during 5 cycles of freeze/thaw.

### Example 2

Aqueous concentrates A and B in accordance with the invention have been characterized in terms of physico-chemical properties and opacifying performances.

They have also been compared to commercially available opacifiers, namely:
Mackadet® OPR1 (available from Solvay), which is an aqueous concentrate containing 15-30% by weight of ethylene glycol distearate, 1-15% by weight of glycerol monostearate, and 1-15% by weight of cocamidopropyl betaine
OPULYN 301 (available from Dow), which is an anionic styrene/acrylic copolymer

Procedures:

Median Diameter

As indicated previously, particle sizes are measured by laser diffraction using laser diffraction granulometer Partica LA-960 (by HORIBA). Size distribution is given by HORIBA LA-960 particle size analyzer. This enable to determine the median diameter of the particles.

The operating conditions were the following:
30 seconds of water degassing is performed by ultrasound before adding the particles to the measuring cell.
The refraction index of the degassed water was 1.33
The ethylene glycol di-stearate refraction index is 1.49.
The water flow in the particle size measuring cell is 2.
The water mixing speed in the particle size measuring cell is 2.

L* Whiteness and Covering Effect

As indicated previously, L* whiteness is determined using the L*a*b* colour space and covering effect is measured by the contrast card method (covering effect (expressed as a %)=(L*_{black background})/(L*_{white background})×100).

Both measurements use the spectrocolorimeter DR LANGE LUCI 100 (measuring geometry d/8° acc. to DIN 5033).

The plates that are used to measure L* whiteness consist of hexagonal shaped cells 2 cm wide by 3 mm thick. The sample quantity for each measurement is 1.5 mL per cell.

Results were the following:

| | Aqueous | Aqueous | | |
|---|---|---|---|---|
| | concentrate A | concentrate B | | |
| | (in accordance | (in accordance | Mackadet | |
| | with the | with the | OPR1 | OPULYN301 |
| Product | invention) | invention) | (comparative) | (comparative) |
| Aspect at room | White liquid | White liquid | White liquid | White liquid |
| temperature (25° C.) | | | | |
| pH | 4.8 | 4.7 | 4.0-6.0 | 2.1-2.5 |
| Active content (%) | 46 | 42 | 41-44 | about 40 |
| Particle size (Horiba | 6 to 10 μm | 2 to 6 μm | 16 to 20 μm | <1 μm |
| granulometer) | | | | |
| RV Viscosity at | about 200 cps | about 200 cps | about 7,500 cps | about 200 cps |
| 10 rpm | | | | |
| L* whiteness | 91.8 | 96.1 | 91.5 | 85.6 |
| Covering effect | 98% | 100% | 97% | 92% |
| L*_{black background} | 91.8 | 96.1 | 91.5 | 85.6 |
| L*_{white background} | 93.6 | 96.1 | 94.6 | 93.5 |

Aqueous concentrate A of the invention exhibits a significantly greater L* whiteness compared to OPULYN 301.

The covering effect measured with aqueous concentrate A of the invention is also significantly greater to the one measured when using OPULYN 301.

### Example 3

The performances of aqueous concentrates A and B in accordance with the invention have also been tested in a simplified personal care composition. The following base body wash composition has been prepared:

34.35 wt % RHODAPEX® ESB 30HA1 (corresponding to 9 wt % of anionic surfactant)

5.00 wt % Mackam® 50ULB (corresponding to 2 wt % of amphoteric surfactant)

0.30 wt % Jaguar® Excel (corresponding to 0.3 wt % of conditioning polymer)

0.63 wt % preservative (0.40 wt % of sodium benzoate and 0.23 wt % of salicylic acid)

1.60 wt % NaCl (salts)

0.50 wt % of citric acid 50% (corresponding to 0.25 wt % of pH adjuster)

Balance Water

To this base body wash composition has been added an opacifier, which was aqueous concentrate A (in accordance with the invention), aqueous concentrate B (in accordance with the invention), Mackadet® OPR1 (comparative) or OPULYN 301 (comparative).

Preparation process was the following

In a plastic beaker, the ingredients are added in the following order.

First, the conditioning polymer is added in water. When it is fully hydrated, then the amphoteric surfactant is added in the main vessel under mechanical stirring (Heidolph RZR2021) with a paddle blade. The anionic surfactant is added afterwards and pH is then adjusted between 4.8 to 5 with the diluted citric acid solution (50% in weight). Preservative (sodium benzoate an salicylic acid) are then added, followed by salts (sodium chloride).

The blend is stirred until an homogeneous mixture is obtained.

To this base body wash composition is then added the opacifier (aqueous concentrate A, or aqueous concentrate B, or Mackadet® OPR1, or OPULYN 301), in an amount of about 1.8% (active material).

The mixture is stirred to obtain a fully opaque and homogeneous solution. pH is adjusted to 4.8-5 with citric acid if need be.

Covering effect and stability upon storage of the corresponding formulations have been tested according to the procedures described previously in Example 1 and 2.

Results were the following:

| | Aqueous | Aqueous | | |
|---|---|---|---|---|
| | concentrate A | concentrate B | | |
| | (in accordance | (in accordance | Mackadet | |
| | with the | with the | OPR1 | OPULYN301 |
| Product | invention) | invention) | (comparative) | (comparative) |
| Dosage in | 1.8 | 1.8 | 1.8 | 0.4 |
| formulation | | | | |
| (% in active) | | | | |
| pH | 4.8 | 4.8 | 4.8 | 4.8 |
| RV Viscosity at | 4260 | 7060 | 3160 | 1180 |
| 10 rpm | | | | |
| (mPa · s⁻¹) | | | | |
| L* whiteness | 59.5 | 65.5 | 36.5 | 37.9 |
| Covering effect | 74% | 79.6% | 47.6% | 47.9% |
| L*_{black background} | 59.5 | 65.5 | 36.5 | 37.9 |
| L*_{white background} | 80.5 | 82.3 | 76.7 | 79.2 |
| Temperature | Stable 3 month | Stable 3 month | Stable 3 month | Stable 3 month |
| storage stability | in temperature | in temperature | in temperature | in temperature |
| (4° C., 40° C., | storage | storage | storage | storage |
| freeze/thaw) | | | | |

A personal care composition containing an aqueous concentrate of the invention (aqueous concentrate A or B) exhibits a significantly greater covering effect compared to an identical personal care composition containing either Mackadet OPR1 or OPULYN 301 as opacifier.

A personal care composition containing an aqueous concentrate of the invention (aqueous concentrate A or B) exhibits a significantly greater Whiteness compared to an identical personal care composition containing either Mackadet OPR1 or OPULYN 301 as opacifier.

### Example 4

In order to illustrate the advantageous high flexibility of the aqueous concentrate of the invention, the following comparative experiment has been carried out.

In a simplified personal care composition, an aqueous concentrate of the invention (Aqueous concentrate A, see Example 1a, in an amount corresponding to 1.8% in active) has been added directly “as is” in order to modify its appearance (in particular increase its opacity).

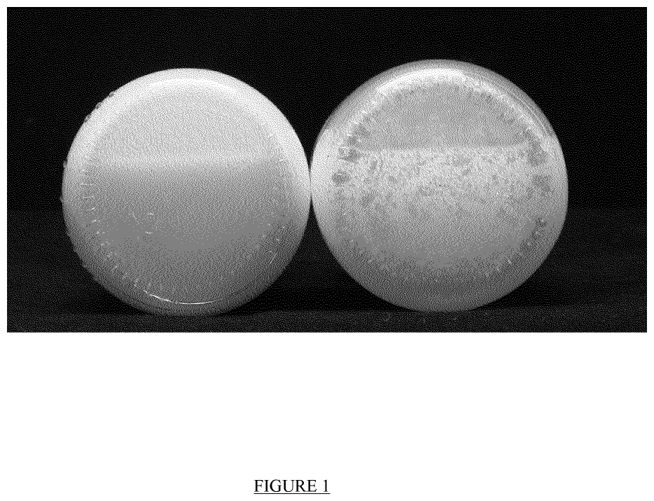

As can be seen on FIG. 1 (composition on the left side), the opacifier particles are readily incorporated into the existing end-use formulation. The resulting formulation is homogeneous.

The same experiment has been conducting using a comparative prior art aqueous concentrate (OPULYN 301, as described previously, in an amount corresponding to 0.4% in active). This comparative aqueous concentrate has also been added directly “as is” in identical relative amount in the same simplified personal care composition.

As can be seen on FIG. 1 (composition on the right side), in this case there is some sedimentation of the opacifier particles. The resulting formulation is not homogeneous. In contrast, part of the opacifier particles tends to deposit on the walls.

This confirm that the opacifier particles of the invention may be readily added into aqueous compositions without any constraints as regards existing manufacturing processes, providing a high flexibility.

Advantageously, aqueous concentrate of the invention may be easily added for example “on top”, namely directly “as is” at the end of the manufacturing process.

The aqueous concentrate of the invention is provided in a liquid form that can be readily used: there is no need to make any premix formulation beforehand.

Also, compared to liquid kaolin-based concentrates of the prior art, the aqueous concentrate of the invention advantageously does not require the presence of rheological agents, or the like, in the final end-use formulation.

This is particularly important in personal care formulation, since such additional rheological agents may negatively impact attributes of said end-use formulation, such as flash foam or sensorial experience.

### Example 5

In order to illustrate the advantageous impact on color intensity of the aqueous concentrate of the invention when added into a coloured composition, the following comparative experiments have been carried out.

The following coloured shampoo compositions have been prepared:

43.56 wt % RHODAPEX® ESB 30HA1 (corresponding to 11.4 wt % of anionic surfactant)

6.20 wt % of colouring agent

5.00 wt % Mackam® 50ULB (corresponding to 2 wt % of amphoteric surfactant)

1 wt % opacifier agent (active ingredient)

0.30 wt % Jaguar® Excel (corresponding to 0.3 wt % of conditioning polymer)

0.63 wt % preservative (0.40 wt % of sodium benzoate and 0.23 wt % of salicylic acid)

1.60 wt % NaCl (salts)

0.60 wt % of citric acid 50% (corresponding to 0.25 wt % of pH adjuster)

Balance of Water

Using 4 different colouring agents (=colouring agent i-iv), a total of 8 coloured shampoo compositions were prepared, namely:
4 compositions including the aqueous concentrate A of the invention (see Example 1) (=“composition of the invention”) and one colouring agent chosen from colouring agent i-iv (=composition i, ii, iii or iv respectively) and
4 comparative compositions including prior art OPULYN 301 (=“comparative composition”) and one colouring agent chosen from colouring agent i-iv (=composition i, ii, iii or iv respectively)

The colouring agents used in each composition were the following:
In composition i: colouring agent=Unicert Violet K7025-J (used in the form of a solution in water at 0.2 wt % of dye)
In composition ii: colouring agent=Unicert Rouge K7057-J (used in the form of a solution in water at 0.2 wt % of dye)
In composition iii: colouring agent=Unicert Rouge 07004-J (used in the form of a solution in water at 0.2 wt % of dye)
In composition iv: colouring agent=Sensient Vert menthe E C0597 (used in the form of a solution in water at 1.0 wt % of dye)

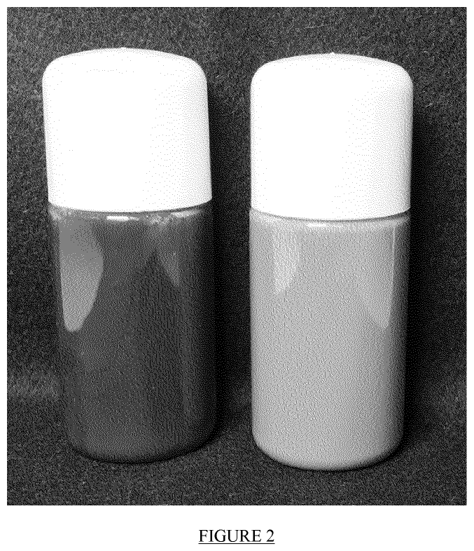

Results can be shown on FIGS. 2-5 showing respectively:

FIG. 2: Compositions (i) containing Unicert Violet K7025-J as colouring agent and either the aqueous concentrate A of the invention (composition of the invention on the left side) or OPULYN 301 (comparative composition on the right side)

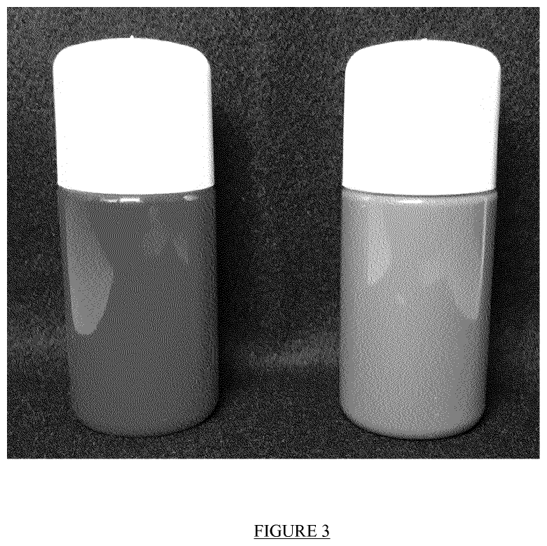

FIG. 3: Compositions (ii) containing Unicert Rouge K7057-J as colouring agent and either the aqueous concentrate A of the invention (composition of the invention on the left side) or OPULYN 301 (comparative composition on the right side)

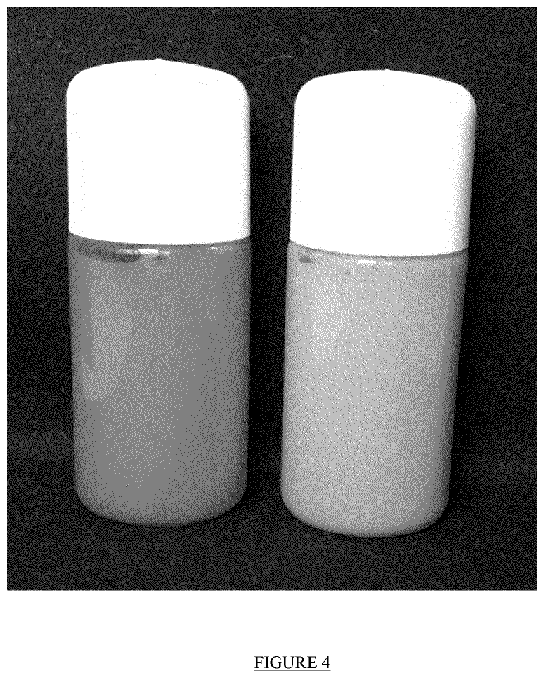

FIG. 4: Compositions (iii) containing Unicert Rouge 07004-J as colouring agent and either the aqueous concentrate A of the invention (composition of the invention on the left side) or OPULYN 301 (comparative composition on the right side)

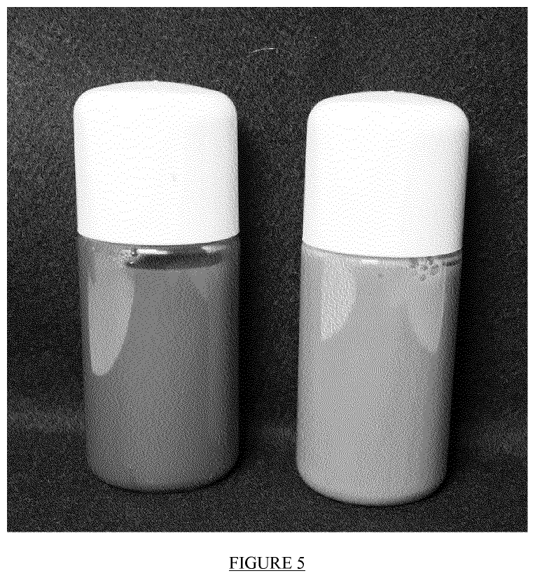

FIG. 5: Compositions (iv) containing Sensient Vert menthe E C0597 as colouring agent and either the aqueous concentrate A of the invention (composition of the invention on the left side) or OPULYN 301 (comparative composition on the right side)

As can be seen on FIGS. 2-5, at iso dose of opacifier particles and whatever the colouring agent, it is visible to the naked eye that a composition of the invention containing the aqueous concentrate A of the invention yields to coloured shampoo compositions that are more intense and more colored compared to comparative compositions containing Opulyn 301.

This demonstrates the benefits in using the aqueous concentrate A of the invention in coloured compositions.

Advantageously, the opacifier concentrates of the invention also do not negatively impact color shade.

Given the specific and improved whiteness of the opacifier concentrates of the invention, coloured compositions containing them do not exhibit greying or blueing effect. This is critical when compared for instance with coloured compositions including Opulyn 301.

### Example 6

The performances of aqueous concentrate B in accordance with the invention has also been tested in an oral care composition.

The following oral care composition (toothpaste chassis) has been prepared (in this example, the amounts correspond of the wt % of the product as is):

0.4 wt % of carboxymethyl cellulose (CMC)

45 wt % of sorbitol

5 wt % of PEG 600

0.8 of wt % sodium fluoride

0.2 wt % of sodium saccharin

10 wt % of Tixosil 63

8.5 wt % of Tixosil 43

0.5 wt % of titanium dioxide

2-5 wt % of surfactants

Flavor

Opacifier (either TiO2 or Aqueous Concentrate B of the invention)

Balance of Water

These formulations have been prepared according to the following procedure:
Prepare a dispersion of CMC in PEG and sorbitol under mixing. Add CMC very slowly, allowing it to fully incorporate before adding water phase.
Prepare a premix of Sodium Fluoride, Sodium Saccharin, and opacifier (either TiO2 or Aqueous Concentrate B of the invention) in Water. Add premix to main batch and mix well.
Add the Tixosil slowly into the main batch under constant mixing.
Add flavor, surfactant and mix slowly until homogenous.

A total of 3 compositions were prepared, using as opacifier either TiO2 (added at 0.5 wt % as is) or an aqueous concentrate of the invention (added respectively at 2 or 4% wt as is).

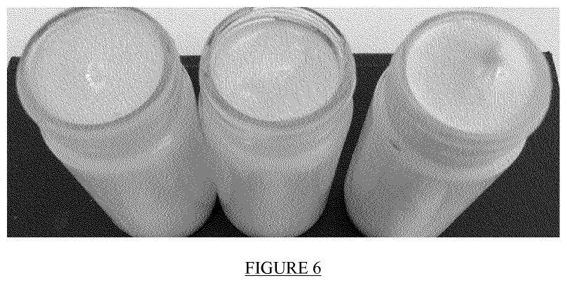

Results can be shown on FIG. 6 showing respectively toothpaste compositions containing aqueous concentrate B of the invention at 2 wt % (on the left side) and at 4 wt % (center) or TiO2 at 0.5 wt % (comparative composition on the right side).

When observed in the bulk, performances of the aqueous concentrate of the invention both in terms of opacity and whiteness are visible to the naked eye.

### Example 7

The performances of aqueous concentrate B have also been tested in a simplified Home care composition.

The following base Hand dish liquid composition has been prepared:

24.28 wt % RHODAPEX® ESB 70 (corresponding to l7 wt % of anionic surfactant)

6 wt % Mackam® 50ULB (corresponding to 2.4 wt % of amphoteric surfactant)

4.44 wt % Mackamine CAO E 36 (corresponding to 1.6% of non ionic surfactants)

0.1 wt % preservative (0.1% Methylisothiazolinone/methyl chloro thiazolinone)

0.6 wt % NaCl (salts)

Balance of Water

To this base Hand Dish liquid composition has been added an opacifier, which was aqueous concentrate B in accordance with the invention.

Preparation process was the following: in a beaker, weight water and then all ingredients in a beaker and mix well until homogeneisation. To this base body wash composition is then added the opacifier (aqueous concentrate B), in an amount of 1.0% (active material). The mixture is stirred to obtain a fully opaque and homogeneous solution.

Covering effect of the corresponding formulations have been tested according to the procedures described previously in Example 1 and 2.

Results were the following:

| | Aqueous concentrate B |
|---|---|
| Product | (in accordance with the invention) |
| Dosage in formulation (% in active) | 1.0 |
| L* whiteness | 47.7 |
| Covering effect | 60.9 |
| L*_{black background} | 47.7 |
| L*_{white background} | 78.4 |

A Home care composition containing the aqueous concentrate B of the invention exhibits improved covering effect and whiteness.

1. A sulfate-free aqueous concentrate comprising at least:
i) at least 25% by weight of opacifier particles selected from C16-C22 fatty acid, C16-C22 fatty alcohol, mono-, di- and tri-C16-C22 esters of glycerol, mono- and di-C16-C22 esters of ethylene glycol, diethylene glycol and triethylene glycol, C16-C22 fatty acid alkanolamides and mixtures thereof, with said particles are having a median diameter measured by laser diffraction ranging from 1 to 15 μm;
ii) an amphoteric or zwitterionic surfactant,
iii) water,
with the total amount of surfactants in said aqueous concentrate ranging from 0.5 to 15% by weight, relative to the total weight of the aqueous concentrate.

2. The aqueous concentrate according to anyone of the preceding claims, wherein said opacifier particles are mono- or di-C16-C22 esters of ethylene glycol and mixtures thereof, for instance ethylene glycol distearate.

3. The aqueous concentrate according to anyone of the preceding claims, wherein said opacifier particles have a median diameter lower than 14 μm, for instance lower than 12 μm and/or greater than 2 μm, for instance greater than 4 μm.

4. The aqueous concentrate according to anyone of the preceding claims, containing at least 30% by weight, for example from 30 to 75% by weight, for example from 30 to 60% by weight, for example from 30 to 50% by weight of said opacifier particles, relative to the total weight of the aqueous concentrate.

5. The aqueous concentrate according to anyone of the preceding claims, containing less than 15% by weight, for example from 0.1 to 15% by weight, for example from 0.5 to 15% by weight, for example from 1 to 10% by weight of said surfactant, relative to the total weight of the aqueous concentrate.

6. The aqueous concentrate according to anyone of the preceding claims, containing at least 20% by weight, for example from 20 to 60% by weight, for example at least 30% by weight, for example at least 40% by weight, for example from 40 to 60% by weight of water, relative to the total weight of the aqueous concentrate.

7. The aqueous concentrate according to anyone of the preceding claims, containing amphoteric or zwitterionic surfactants in an amount ranging from 0.5 to 15% by weight, especially from 1 to 10% by weight, for instance from 4 to 8% by weight, relative to the total weight of the aqueous concentrate.

8. The aqueous concentrate according to anyone of the preceding claims, containing from 0 to less than 2% by weight of non ionic surfactant relative to the total weight of the aqueous concentrate.

9. The aqueous concentrate according to anyone of the preceding claims, containing from 0 to less than 2% by weight of anionic surfactant relative to the total weight of the aqueous concentrate

10. The aqueous concentrate according to anyone of the preceding claims, containing from 0 to less than 2% by weight of emollient, such as for instance glyceryl oleate, relative to the total weight of the aqueous concentrate.

11. Use of the aqueous concentrate of claim 1 as an opacifier.

12. A method for modifying the appearance of a composition, comprising adding an aqueous concentrate according to claim 1 to such composition.

13. A method to increase the opacity and/or whiteness of a composition, comprising adding an aqueous concentrate according to claim 1 to such composition.

14. A composition comprising from 1 to 5% by weight of the aqueous concentrate of claim 1, relative to the total weight of the composition.

15. The composition of claim 14, further containing a colouring agent.
